(19) United States
(12) Patent Application Publication
(10) Pub. No.: US 2005/0148919 A1
(43) Pub. Date: Jul. 7, 2005

(54) Device for passive mobilization of the ankle

(76) Inventors: Laurent Beny, Charroue (FR); Jose Andry, Bourg Fidele (FR)

Correspondence Address: CLARK & BRODY, 1090 VERMONT AVENUE, NW, SUITE 250, WASHINGTON, DC, 20005, US

(21) Appl. No.: 10/961,096

(22) Filed: Oct. 12, 2004

(30) Foreign Application Priority Data
Oct. 9, 2003 (FR) 03 11 841

(51) Int. Cl. A61F 5/00

(52) U.S. Cl. 602/28

(57) ABSTRACT

According to the invention, the mobilisation device at least comprises: a base plate (2), and a tibial support (7) integral with the base plate (2) and whose longitudinal axis extends over a sagittal plane (S). A foot support assembly (10) that is integral with the base plate (2) being substantially aligned with the tibial support (7) comprises a cradle (20) mobile in rotation about an axis (Δ) parallel to the sagittal plane (S), so as to allow user movements of foot abduction and adduction, and a footrest plate (21) integral with the cradle (20) and mobile in rotation with respect to the cradle (20) about an axis (Δ′) forming an acute, nonzero angle (α) with the rotation axis (Δ) of the cradle (20), so as to allow user movements of foot inversion and eversion. A motorised driving means (15) is provided for driving the cradle (20) in alternate rotation about its axis (Δ), intended to induce at least alternate passive movements of foot adduction and abduction by the device user

The present invention concerns the technical area of devices enabling the functional rehabilitation of a lower limb, and more particularly the ankle joint.

In this area, passive mobilisation devices are known used to rehabilitate the different movements of the ankle, such as foot flexion-extension or adduction-extension, or inversion-eversion movements of the foot that are achieved passively for the patient i.e. with no muscle action.

Said passive mobilisation devices, such as those marketed by ABILITYONE under reference KINETEC 5190 CPM, are particular in that they use a specific motorisation for each of the above-mentioned movements. Therefore this kind of passive mobilisation device comprises a motor dedicated to flexion-extension motion of the foot, a motor dedicated to abduction-adduction motion and a further motor for inversion-eversion motion of the foot. These motors are piloted by an automatic control system, in such manner as to coordinate their functioning in relation to the desired rehabilitation movements.

This type of device is fully satisfactory in respect of the objectives of functional rehabilitation of the ankle via passive mobilisation, but has the disadvantage however of being relatively costly on account of the complexity of its kinematics and the required automatic control system to ensure optimum functioning.

The need has therefore arisen for a new type of passive mobilisation device for the ankle joint which is able to ensure passive mobilisation of this joint, in particular the combined motions of foot abduction-eversion and adduction-inversion, which requires fewer motorisation members to obtain the desired movements and hence provides a passive mobilisation device for the ankle joint having simplified kinematics and therefore of lower cost price than passive ankle mobilisation devices of the prior art.

To achieve this objective, the invention concerns a passive mobilisation device for the ankle joint, wherein it comprises:
a base plate,
a tibial support integral with the base plate and whose longitudinal axis extends over a sagittal plane,
a foot support assembly integral with the base plate being substantially aligned with the tibial support and comprising:
   a cradle rotationally mobile along an axis parallel to the sagittal plane, to allow abduction and adduction motion of the user's foot,
   a footrest plate integral with the cradle and rotationally mobile with respect to the cradle along an axis forming a non-zero, acute angle with the rotation axis of the cradle, to allow inversion and eversion motion of the user's foot,
and motorised means for driving the cradle in alternate rotation about its axis, intended to induce at least alternate passive adduction and abduction motion of the device user's foot.

The form of the foot support assembly of the invention has the advantage, through simple alternate rotation of the cradle, of ensuring the rehabilitation of adduction and abduction motion, whilst allowing induced inversion and eversion motion. Therefore, with one single motor it is possible to obtain functional rehabilitation of the user's ankle by means of complex movements inducing two degrees of freedom in rotation. The design of the passive mobilisation device of the invention therefore permits the achieving of functional rehabilitation of the ankle joint by means of a single rotary motorisation member, whereas hitherto it was necessary to use at least two rotary motorisation members operating along two separate axes.

According to one fabrication characteristic, in order to better conform to the anatomical axes of the ankle joint, the axis of rotation Δ′ of the footrest with respect to the cradle is substantially parallel to a plantar bearing plane while being distant from this bearing plane.

According to another characteristic of the invention, again for better conformity with the anatomical articulation axes of the rehabilitated member, the axis of rotation Δ′ of the footrest and the axis of rotation Δ of the cradle, projecting into the sagittal plane, form an angle α of between 70° and 90°.

According to a further characteristic of the invention, the device preferably, but this is not strictly necessary, comprises means for connecting the footrest to a member integral with the base plate. These connection means are then adapted so that rotation of the cradle in the direction of adduction causes the footrest to rotate about its axis Δ′ in the direction of an inversion, whilst a rotation of the cradle in the direction of an abduction, causes the footrest to rotate in the direction of an eversion.

This advantageous characteristic of the invention therefore allows the passive prompting of the combined foot movements of adduction inversion and abduction eversion by the device user. This means that with a single motorisation axis in alternate rotation, it is possible to obtain passive, forced mobilisation of the ankle joint in two separate rotation axes.

According to the invention, the connection means, between the cradle and the base plate, may be formed in any appropriate manner, insofar as they guarantee the aforesaid combinations of movements.

Therefore, the connection means may be made in the form of at least one flexible link, such as a cable, connecting the cradle to part of the base plate that is fixed with respect to the cradle, during adduction and abduction movements of the said cradle.

According to one preferred, but not strictly necessary, characteristic of the connection means, these include a connecting rod linked via one of its ends to the footrest and at its other end to a member integral with the base plate. In a preferred embodiment, the connecting rod is then connected to the member integral with the base plate via an annular or cylindrical-spherical link.

According to another characteristic of the invention, the connecting rod is linked to the footrest by a pivot link having an axis perpendicular to rotation axis Δ′ of the footrest.

According to a further characteristic of the invention, and in order to ensure optimum functional rehabilitation of the ankle joint, the means connecting the footrest to the base plate or to a member of the latter that is fixed with respect to the cradle, are adapted to achieve alternate rotation of the footrest about its axis Δ′, with a range of motion about a so-called rest position of between 10° and 20°, or a full rotation between maximum inversion and maximum eversion of between 20° and 40°.

According to a still further characteristic of the invention, again for the purpose of ensuring optimum functional rehabilitation of the ankle, the driving means are adapted to ensure adduction and abduction over a range of motion with respect to a rest position of between 10° and 30°, or a range of motion between a maximum adduction position and a maximum abduction position of between 20° and 60°.

According to a preferred, but not strictly necessary, embodiment of the invention, the driving means comprise a gear motor unit which is integral with the base plate and which has a drive shaft of axis γ on which the cradle of the foot support assembly is fixed, so that rotation axis Δ of the cradle merges with axis γ of the drive shaft. Therefore, the gear motor unit is adapted so that its axis γ is substantially aligned with the tibial support being parallel to the sagittal plane. With this embodiment, it is possible to obtain a device of the invention that is relatively compact, with a driving mechanism of simple design and small bulk.

According to a further characteristic of the invention, and to allow several modes of use of the passive mobilisation device for the ankle joint to achieve functional rehabilitation both of abduction-adduction motion and of foot flexion-extension, the cradle of the foot support assembly is fixed removable fashion onto the gear motor unit, whilst the gear motor unit is adapted onto carrier means whose angular position with respect to the base plate is adjustable, so that it is possible to place axis γ of the drive shaft either parallel to the sagittal plane to ensure the motorisation of adduction abduction motion, or perpendicular to the sagittal plane to ensure motorisation of foot flexion-extension motion.

In preferred, but not strictly necessary, manner the device of the invention then includes a foot support assembly intended to be connected to the drive shaft of the gear motor unit when the drive shaft is perpendicular to the sagittal plane, so as to mobilise the ankle in said movements of foot flexion and extension.

According to a further characteristic of the invention, the driving means and the gear motor unit are adapted to control flexion extension movements having a range of motion, with respect to a mean position, of between 10° and 60° or so that they allow movement between a maximum foot flexion position and a maximum foot extension position with a range of motion of between 50° and 80°.

Evidently, in relation to the pathologies of the device user, it is possible to provide for ranges of motion that are different to those mentioned above.

According to a still further characteristic of the invention, to allow use of the passive mobilisation device for the ankle joint in different user positions, the tibial support and the foot support assembly, optionally associated with the gear motor unit, are supported by a chassis which is integral with the base plate and whose angle of incline with respect to the base plate is adjustable to allow use of the device when the user is in sitting or lying position. Various other characteristics of the invention will become apparent from the description below made with reference to the drawings which illustrate a preferred, but non-restrictive, embodiment of a passive mobilisation device for the ankle joint according to the invention.

FIG. 1 is a perspective, three-quarter, right front view of a passive mobilisation device of the invention.

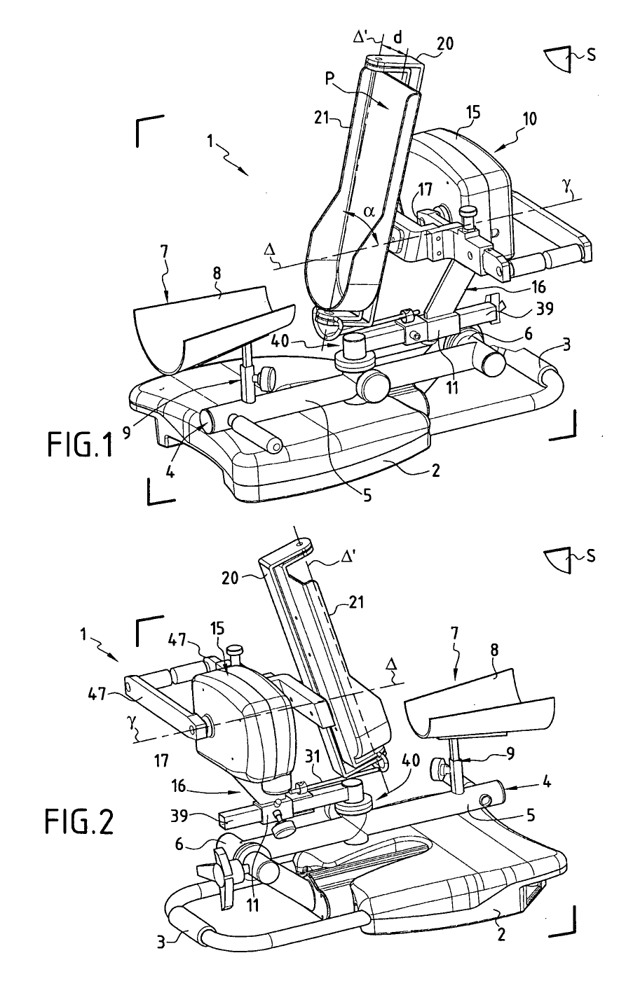

FIG. 2 is a perspective, three-quarter left rear view of the device shown in FIG. 1.

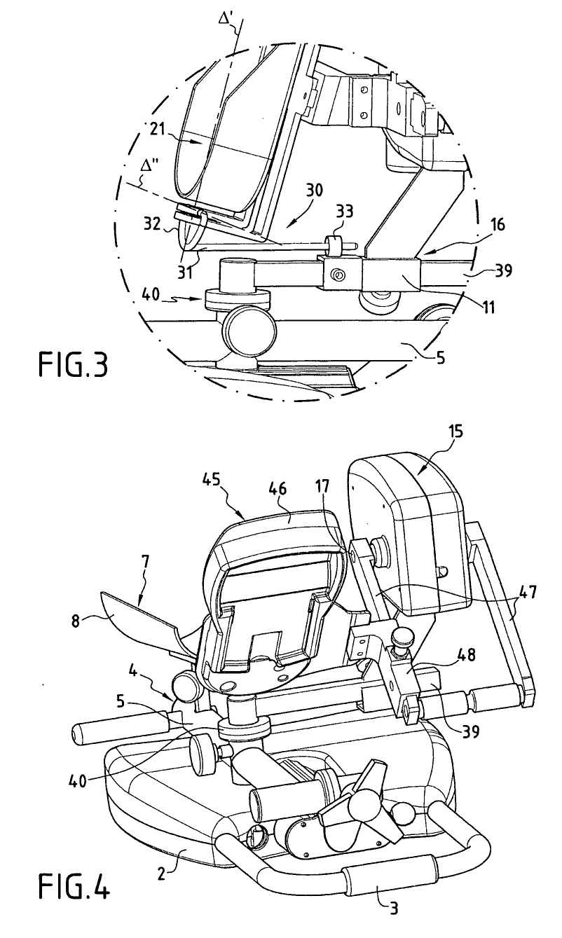

FIG. 3 is a perspective view showing a detail of a preferred embodiment of the device of the invention.

FIG. 4 is a rear perspective of the device, such as illustrated in FIG. 1, in another configuration of use.

In a preferred, but non-exclusive, embodiment, a passive mobilisation device of the ankle joint according to the invention, such as illustrated in FIGS. 1 and 2 and generally denoted 1, comprises a base plate 2 intended to be laid on a support such as the floor or a bed. According to the illustrated example, base plate 2 is provided with a carry handle 3, intended to facilitate handling and moving of the base plate 2.

According to the illustrated example, the base plate 2 carries a chassis 4 which here is in the form of a single spar 5 which, at one of its ends, is fixed to base plate 2 via a locking pivot 6 having a horizontal axis, allowing adjustment of the angle formed, in a sagittal plane S, by chassis 4 and base plate 2. It is to be noted that if, in the illustrated example, the chassis 4 comprises a single spar 5, it could be made in any other form such as a frame with two parallel spars.

The chassis 4, close to its end opposite the locking pivot 6, is fitted with a tibial support 7 which is therefore integral with base plate 2 via the chassis 4. According to the illustrated example, the tibial support 7 comprises a semi-shell 8 intended to receive the calf of the user of device 1, the position of the semi-shell 8 then being height adjustable with respect to the chassis 4, via a telescopic arm 9.

Device 1 also comprises a foot support assembly 10 for the user of the device. The foot support assembly 10 is adapted onto chassis 4, via a slide 11, so that the distance between the tibial support 7 and the foot support assembly 10 is adjustable. Evidently, the use of slide 11 is not strictly necessary for producing a device of the invention, insofar as the position of the foot support assembly 10 could be fixed on chassis 4 or even on base plate 2. In this case, there is no provision for adjusting the distances between tibial support 7 and the foot support assembly 10, or this distance could be adjusted by moving the tibial support 7 on chassis 4.

In the illustrated example, the foot support assembly 10, as motorised driving means, comprises a gear motor unit 15 which is fixed to the slide 11 via a bracket 16. The gear motor unit 15 comprises a drive shaft 17 whose axis γ lies substantially in the alignment of the tibial support 7 being at least parallel to the sagittal plane S and is here contained within the sagittal plane S.

According to one essential characteristic of the invention, the foot support assembly 10 also comprises a cradle 20 which, in the illustrated example, is connected to the shaft 17, so that the cradle 20 is mobile in rotation about an axis Δ merging with axis γ and parallel to the sagittal plane S, to allow for abduction or adduction motion of the user's foot, as will be seen below. According to a further essential characteristic of the invention, the foot support assembly 10 also comprises a footrest plate 21 which is integral with the cradle, being mobile with respect to the latter along an axis Δ′ substantially co-planar with axis Δ.

In preferred, but not strictly necessary, manner rotation angle Δ′ of the footrest plate 21 and rotation angle Δ of the cradle, projecting into the sagittal plane S, together form an angle α of between 70° and 90°.

Device 1 for passive mobilisation of the ankle joint so designed therefore permits functional rehabilitation of the ankle joint both in foot adduction and abduction, and in inversion and eversion motion of the same foot. The gear motor unit 15, through the driving of shaft 17, enables rotation of the cradle 20 and hence of the footrest plate 21 about axis Δ through a motion, according to direction, corresponding either to foot abduction or to foot adduction. Conjointly, the articulation of the footrest 21 about axis Δ′ enables associated motion of foot inversion or eversion.

It is to be noted that, in preferred manner, axis Δ′ lies substantially parallel to the plane P of plantar bearing of the footrest plate 21, while being offset with respect to the latter so as to better correspond to the anatomical axes of articulation of the user's ankle. In preferred, but not strictly necessary, manner the oscillation axis Δ′ of the footrest plate is then positioned at a distance d from the plane of plantar bearing P measuring between 20 mm and 60 mm.

With the passive mobilisation device 1, such as described above, it is possible to impose abduction and adduction motion upon the user's foot. However, it is not certain that these movements are associated with the corresponding movements of inversion and eversion.

Therefore, according to a preferred embodiment of the invention, in order to permit effective prompting of inversion and eversion movements, the foot support assembly 10, as shown in particular FIG. 3, comprises connection means 30 to connect the footrest 21 to a member integral with the base plate 2, which in the illustrated example consists of bracket 16. In the meaning of the invention, the connection means 30 must connect the footrest 21 to a part of the device 1 which is fixed during movements of adduction abduction of cradle 20.

According to the illustrated example, the connection means 30 comprise a connecting rod 31 of which a first end is fixed via a fork 32, to the footrest 21. It is to be noted that the fork 32 is adapted to the footrest 21, so that it can pivot about an axis Δ″ perpendicular to rotation axis Δ′ of the footrest 21. The end of the connecting rod 31, that is opposite fork 32, is fixed to bracket 16, via a ring 33 which, with connecting rod 31, defines an annular link, also called a cylindrical-spherical link. Therefore, the connecting rod 31 has two degrees of freedom in rotation and one degree of freedom in translation with respect to bracket 16.

The provision of connection means 30 makes it possible, in highly advantageous manner, to induce a movement of foot inversion during an adduction movement of the cradle, and conversely a movement of foot eversion during an abduction movement of the cradle. This advantageous provision of the invention guarantees perfect prompting of the combined movements of adduction-inversion and abduction-eversion of the foot and hence good functional rehabilitation of the ankle.

According to a further characteristic of the invention that is preferred but not strictly necessary, the slide 11 is carried by an arm 39 fixed to chassis 4 by a locking pivot 40 whose axis is parallel to the sagittal plane S and perpendicular to axis γ of shaft 17. Therefore, the locking pivot 40 allows arm 39 and hence shaft 17 to be placed in two positions, namely such as illustrated FIG. 1, a position in which the shaft 17 is substantially aligned with the tibial support 7 and parallel to the sagittal plane S and, a second position, such as illustrated in particular FIG. 4, in which the shaft 17 lies substantially perpendicular to the sagittal plane S. Consequently, the gear motor unit 15 may be used for mobilisation of the ankle joint in movements of foot flexion and extension. For this purpose, the foot support assembly 10, such as described above, can be dismounted and another foot support assembly 45 is adapted onto shaft 17 such as shown FIG. 4.

This second support assembly 45 consists of a simple footrest 46 which is joined to shaft 17 by means of an arm 47 and a slide 48 with which to adjust the distance between the footrest 46 and shaft 17.

It is to be pointed out that, according to an advantageous characteristic of the invention, the gear motor unit 15 has two arms 47, each fixed to an end of shaft 17, either side of the gear motor unit 15. With this arrangement it is possible to use the gear motor unit 15 for passive mobilisation in flexion-extension of the joint, either for the user's right ankle or left ankle.

It is to be pointed out also that the first foot support assembly 10, intended for mobilisation and rehabilitation of abduction-adduction movements, is connected to shaft 17 firstly via one of the two arms and secondly via an adaptor device 50 designed to permit alignment of the rotation axis of cradle 20 with the tibial support means 7.

Evidently, said embodiment of the connection between the cradle 20 and the shaft 17, is not strictly necessary for obtaining a passive mobilisation device for the ankle joint, and another mode of fixation could be considered.

Various other modifications could be made to the invention without departing from the scope thereof.

1. Passive mobilisation device for the ankle joint of a person using the device, wherein it at least comprises:
a base plate (2),
a tibial support (7) integral with the base plate (2) and whose longitudinal axis extends over a sagittal plane (S),
a foot support assembly (10) that is integral with the base plate (2) being substantially aligned with the tibial support (1) and which comprises:
a cradle (20) mobile in rotation about an axis (Δ) of adduction/abduction parallel to the sagittal plane (S), so as to allow user movements of foot adduction and abduction,
a footrest plate (21) integral with the cradle (20) and rotationally mobile with respect to the cradle (20) about an axis (Δ′) forming an acute, nonzero angle (α) with the axis of rotation (Δ) of the cradle (20), so as to allow inversion and eversion motion of the user's foot
and motorised means (15) for driving the cradle (20) in alternate rotation about its axis (Δ), intended to induce at least alternate passive movements of foot adduction and abduction of the device user.

2. Device as in claim 1, wherein the rotation axis (Δ′) of the footrest (21) with respect to the cradle (20) is substantially parallel to a plane of plantar bearing (P), being distant from this plane of bearing (P).

3. Device as in claim 1, wherein the axis of rotation (Δ′) of the footrest and the axis of rotation (Δ) of the cradle, when projected into the sagittal plane (S), form an angle (α) lying between 70° and 90°.

4. Device as in claim 1, wherein it comprises connection means (30) connecting the footrest (21) to a member (33) integral with the base plate (2), which connection means (30) are adapted so that rotation of the cradle (20) in the direction of an adduction causes rotation of the footrest (21) about its axis (Δ′) in the direction of an inversion, whilst a rotation of the cradle (20) in the direction of an abduction causes rotation of the footrest (21) in the direction of an eversion.

5. Device as in claim 4, wherein the connection means connecting the footrest (21) to the base plate (2) are adapted to cause alternate rotation of the footrest (21) about its axis (Δ′) whose range of motion about a so-called rest position, lies between 10° and 20°.

6. Device as in claim 4, wherein the connection means (30) connecting the footrest (21) to the base plate (2) comprise a connecting rod connected at one of its ends to the footrest (21) and at its other end to member (33) integral with the base plate (2).

7. Device as in claim 6, wherein the connecting rod (31) is connected to member (33) integral with base plate (2) via an annular or cylindrical-spherical connection.

8. Device as in claim 6, wherein the connecting rod (31) is connected to the footrest (21) via a pivot link whose axis (Δ″) is perpendicular to rotation axis (Δ′) of the footrest (21).

9. Device as in claim 1, wherein the driving means (15) are adapted to ensure movements of adduction and abduction having a range of motion with respect to a rest position, of between 10° and 30°.

10. Device as in claim 1, wherein the driving means (15) comprise a gear motor unit which is integral with the base plate (2) and which has a drive shaft (17) of axis (γ) on which the cradle (20) is fixed, so that rotation axis (Δ) of the cradle merges with axis (γ) of the drive shaft.

11. Device as in claim 10, wherein:
the cradle (20) is fixed removable fashion onto the gear motor unit (15),
the gear motor unit (15) is adapted onto an arm (39) whose angular position with respect to the base plate can be adjusted so that it is possible to position axis (γ) of the drive shaft (17), either parallel to the sagittal plane (S), or perpendicular to the sagittal plane (S).

12. Device as in claim 11, wherein it comprises a foot support assembly (45) intended to be connected to the drive shaft (17) of the gear motor unit (15), when the axis (γ) of the drive shaft (17) is perpendicular to the sagittal plane (S), so as to mobilise the ankle in movements of foot flexion and extension.

13. Device as in claim 1, wherein the tibial support (7) and the foot support assembly (10) are carried by a chassis (4) that is integral with the base plate (2) and whose angle of incline, in the sagittal plane (S) with respect to the base plate can be adjusted so as to permit use of the device in user sitting or lying position.
